SEQUENCE LISTING (16 sequences)

SEQ ID NO: 1 - DNA, 73 bp, Artificial Sequence
  Synthetic 5′UTR leader sequence
acacgtatttttacaacaataccaacaacaacaacaacaaacaacattacaattacgtatttctctctctaga

SEQ ID NO: 2 - DNA, 54 bp, Artificial Sequence
  Synthetic primer
gatatctccactgacgtaagggatgacgcacaatcccactatccttcgcaagac

SEQ ID NO: 3 - DNA, 54 bp, Artificial Sequence
  Synthetic primer
atgaaatgaacttccttatatagaggaagggtcttgcgaaggatagtgggattg

SEQ ID NO: 4 - DNA, 53 bp, Artificial Sequence
  Synthetic primer
tctatataaggaagttcatttcatttggagagaacacgtatttttacaacaat

SEQ ID NO: 5 - DNA, 52 bp, Artificial Sequence
  Synthetic primer
taatgttgtttgttgttgttgttgttggtattgttgtaaaaatacgtgttct

SEQ ID NO: 6 - DNA, 53 bp, Artificial Sequence
  Synthetic primer
aacaacaacaacaaacaacattacaattacgtatttctctctctagaggatcc

SEQ ID NO: 7 - DNA, 19 bp, Artificial Sequence
  Synthetic primer
cacccggggatcctctaga

SEQ ID NO: 8 - DNA, 25 bp, Artificial Sequence
  Synthetic primer
acaattacgtatttctctctctaga

SEQ ID NO: 9 - DNA, 21 bp, Artificial Sequence
  Synthetic primer
cgatcggggaaattcgagctc

SEQ ID NO: 10 - DNA, 22 bp, Artificial Sequence
  Synthetic primer
ttacgctgaagagatgctcgac

SEQ ID NO: 11 - DNA, 25 bp, Artificial Sequence
  Synthetic primer
cctaaagagaggttaaagccgacag

SEQ ID NO: 12 - DNA, 20 bp, Artificial Sequence
  Synthetic primer
acatccaaggaaggcagcag

SEQ ID NO: 13 - DNA, 26 bp, Artificial Sequence
  Synthetic primer
gactcatagagcccggtattgttatt

SEQ ID NO: 14 - DNA, 65 bp, Artificial Sequence
  Synthetic 5′UTR leader sequence
acacgtatttttacaacaataccaacaacaacaacaacaaacaacattgtatttctctctctaga

SEQ ID NO: 15 - DNA, 65 bp, Artificial Sequence
  Synthetic 5′UTR leader sequence
acacgtagttttacaacaataccaacaacaacaacaacaaacaacagtgtagttctctctctaga

SEQ ID NO: 16 - DNA, 76 bp, Artificial Sequence
  Synthetic 5′ UTR leader sequence
acacgtagttttacaacaataccaacaacaacaacaacaaacaacaacaacaacaacaagtagttctctctctaga

(19) United States
(12) Patent Application Publication
(10) Pub. No.: US 2015/0284738 A1
(43) Pub. Date: Oct. 8, 2015

(54) ARTIFICIAL DNA SEQUENCE WITH OPTIMIZED LEADER FUNCTION IN 5' (5'-UTR) FOR THE IMPROVED EXPRESSION OF HETEROLOGOUS PROTEINS IN PLANTS

(71) Applicant: RODINA HOLDING S.A., LUGANO (CH)

(72) Inventors: Stefano MARCHETTI, Pagnacco (UD) (IT); Francesca DE AMICIS, Buttrio (UD) (IT); Tamara PATTI, Buja (UD) (IT)

(21) Appl. No.: 14/744,221

(22) Filed: Jun. 19, 2015

Related U.S. Application Data
(63) Continuation-in-part of application No. 12/521,355, filed on Jun. 26, 2009, now Pat. No. 9,085,775.

(30) Foreign Application Priority Data
Dec. 29, 2006 (IT) UD2006A000280

(51) Int. Cl. C12N 15/82; C12N 15/113

(52) U.S. Cl. CPC C12N 15/8257; C12N 15/113

(57) ABSTRACT

A nucleotide leader sequence 5′-UTR comprises elements favorable to gene expression, such as repeated CAA trinucleotide elements in combination with repeated CT dinucleotide elements.

### CROSS-REFERENCE TO RELATED APPLICATIONS

This Application is a Continuation-in-Part of U.S. patent application Ser. No. 12/521,355, filed Jun. 26, 2009, which is a national stage application (under 35 U.S.C. §371) of PCT/EP2007/064590, filed Dec. 27, 2007, which claims benefit of Italian Application No. UD2006A000280, filed Dec. 29, 2006, the entire contents of each of which are incorporated by reference herein.

### FIELD OF THE INVENTION

The present invention concerns an artificial DNA sequence for improving the expression of heterologous proteins in plants.

### BACKGROUND OF THE INVENTION

In the field of biotechnology, there is a strongly felt need to enhance the level of expression of genes introduced into the relative organisms. This level is often unsatisfactory, and represents a barrier to the industrial application of innovations in plant and animal biotechnology. There is a quantity of data to support the importance of the leader region in regulating the levels of gene expression, while there are various structural elements that characterize the regulation capacity thereof.

In this case, the untranslated leader sequence in 5′ (5′-UTR), as it is proposed in widely diffused vectors (e.g. pBI121 and derivatives, pCAMBIA and derivatives), has numerous defects which make it unsuitable to direct adequate levels of gene expression in genetically modified organisms. In particular, when yields are to be maximized (e.g. the use of plants as cellular factories for compounds useful to man), it is necessary to eliminate the production constraints exerted by the 5′-UTR sequence. To this end, the leader Ω (a sequence that exists naturally in tobacco mosaic virus, TMV) has been proposed in plants. However, this too has imperfections and redundancies that render it open to improvement.

It is known that the region poly(CAA) in the translational enhancer present in the leader Ω of TMV (Gallie and Walbot 1992 Nucleic Acids Res., 20, 4631-4638) significantly enhances the expression levels, that is, it has a positive effect on the translation levels of heterologous proteins in vitro and in vivo (Gallie et al. 1988a Nucleic Acids Res., 16, 883-893, Gallie et al. 1988b Nucleic Acids Res., 16, 8675-8694, Gallie 2002 Nucleic Acids Res., 30, 3401-3411). In the leader Ω, a poly(CAA) sequence is associated with 3 repeats of the sequence ACAATTAC (Gallie et al. 1988a), but delection studies have shown that the regulator element responsible for enhancing the expression levels may consist of a single copy of the sequence ACAATTAC in combination with the motif (CAA)n (Gallie and Walbot 1992).

It is also known that the transcription initiation site (Inr) of the CaMV 35S promoter (Guilley et al. 1982 Cell, 30, 763-773) favours an efficient capping of the mRNA. Furthermore, it is known that many plant leaders (Bolle et al. 1996 Plant Mol. Biol. 32, 861-868) have a sequence rich in CT elements and that the CT-rich sequences can alter the transcription levels (Chen et al. 1996 J. Virol., 70, 8411-8421).

It is also known that sequences which have a length of more than 40 nucleotides promote the recognition of the first AUG as authentic initial translation codon (Kozak 1989 J. Cell. Biol., 108, 229-241). For example, it has been observed that the extension of the leader from 29 to 74 nt causes an increase in the translation level of mRNA in vitro (Kozak 1991, J. Biol. Chem., 266, 19867-19870) and in vivo (Gallie and Walbot 1992). Leader sequences with a greater content of A/T cause higher expression levels since the formation of segments of double strand mRNA, due to the folding of the molecule over itself, is discouraged. In fact, it is certain that such secondary structures have a depressing effect on the translation efficiency (Pelletier and Sonenberg 1985 Cell, 40, 515-526; Kozak 1986 Proc. Natl. Acad. Sci. USA, 83, 2850-2854). Moreover, it has been noticed that the introduction of portions of 5′-UTRs of viral origin into plant leaders can determine an increase in the level of expression of reporter proteins (Dowson Day et al. 1993 Plant Mol. Biol., 23, 97-109).

Purpose of the present invention is therefore to obviate the shortcomings of the state of the art and to achieve a leader sequence that increases the expression levels of recombinant proteins in plants.

The Applicant has devised, tested and embodied the present invention to overcome the shortcomings of the state of the art and to obtain these and other purposes and advantages.

### SUMMARY OF THE INVENTION

The present invention is set forth and characterized in the independent claims, while the dependent claims describe other characteristics of the invention or variants to the main inventive idea.

In accordance with the above purpose, an artificial DNA sequence having a leader function in 5′ (5′-UTR), hereafter indicated by LL-TCK, according to the present invention simultaneously comprises elements favorable to gene expression, such as repeated trinucleotide elements CAA in combination with repeated dinucleotide elements CT.

The LL-TCK sequence according to the present invention was obtained by means of artificial synthesis and is the fruit of the intellect, since it does not exist in nature. According to an advantageous solution, the LL-TCK sequence according to the present invention provides the combination of trinucleotide elements CAA with dinucleotide elements CT and a modification of the sequences that activate translation present in the Ω leader.

According to a variant, the sequence according to the present invention contains a poly(CAA) region, that is, an oligonucleotide consisting of 2 or more copies of the CAA element, preferably but not necessarily contiguous with each other.

According to another variant, the sequence according to the present invention contains a poly(CT) region, that is, an oligonucleotide consisting of 2 or more copies of the CT element, preferably but not necessarily contiguous with each other.

A variant of the present invention provides that the sequence contains one or more copies of the octamer ACAATTAC.

A sequence obtained from the combination of the sequences with a poly(CAA) region and those with a poly(CT) region also comes within the field of the present invention.

A sequence obtained from the combination of the sequences with a poly(CAA) region and those with one or more copies of the octamer ACAATTAC also comes within the field of the present invention.

Furthermore, a sequence obtained from the combination of the sequences with a poly(CT) region and those with one or more copies of the octamer ACAATTAC also comes within the field of the present invention.

According to the present invention, it is possible to provide a sequence obtained from the combination of the sequences with a poly(CAA) region, those with a poly(CT) region and those with one or more copies of the octamer ACAATTAC.

Furthermore, again according to the present invention, it is possible to provide a sequence obtained from the combination of one or more of the above sequences with the CaMV 35S Inr site, that is, the transcription initiation site of the cauliflower mosaic virus 35S promoter.

The LL-TCK sequence according to the present invention is thus able to increase the expression levels of heterologous proteins in transgenic plants. According to an advantageous solution of the present invention, the new sequence LL-TCK was synthesized so as to create a combination of the following elements according to an original pattern, unique of its kind:
(1) transcription start site (Inr) of the CaMV 35S promoter for an efficient mRNA capping;
(2) poly(CAA) region similar to the translational enhancer present in the TMV Ω leader;
(3) a sequence rich in CT elements, like many plant leaders.

Furthermore, the LL-TCK sequence has a length of more than 40 nucleotides in order to promote the recognition of the first AUG as the authentic translation start codon (Kozak 1989) and an overall content of G+C of less than 40%.

According to a particular solution of the present invention, the LL-TCK sequence is the one shown in SEQ ID NO: 1 (5′-3′).

It is possible to foresee that small mutations in the LL-TCK sequence do not alter its effectiveness and for this reason the present invention also refers to leader sequences derived from the present sequence, for example following deletion or duplication of a CAA triplet, substitution or deletion of a single base, etc.

The innovation of LL-TCK consists in the fact that it joines in a single leader a modified poly(CAA) element, an octamer from the TMV Ω leader and a CT-rich sequence of plant origin.

Therefore, the artificial sequence LL-TCK according to an advantageous solution of the present invention provides the presence of a single octamer ACAATTAC associated with 9 CAA repetitions located in position 5′ with respect to the octamer.

Since the ATT triplet inside the element ACAATTAC can represent a non-canonical translation start site (Tyc et al. 1984 Eur. J. Biochem., 140, 503-511, Schmitz et al. 1996 Nucleic Acids Res., 24, 257-263), in the LL-TCK leader this triplet has been put in frame with a stop codon.

Furthermore, in the artificial LL-TCK leader an element (CT)₄ has been added to the 3′ end of the regulator element obtained from the union of the poly(CAA) sequence with the octamer ACAATTAC. The combination of these two elements, for each of which the positive effect on gene expression is known, has never been found in nature or previously made by man.

The LL-TCK leader, combining these two elements, causes an enhancement of both the translation level and the transcription level of the gene concerned.

This effect has been demonstrated by comparing the expression levels of the gene uidA obtained in tobacco plants (Nicotiana tabacum) transformed with the constructs 35S-LL-TCK:: uidA (pSTART) and 35S::uidA (pBI121 with original leader). The vector pSTART was obtained by replacing the leader sequence in pBI121 (Clontech) with LL-TCK. In particular, the object of the replacement and manipulation was the pB121 sequence comprised between the region Inr (ACACG) and the restriction site Xba 1 (TCTAGA). The nucleotide sequence flanking the initial translation codon of uidA was kept unvaried in the two constructs so as to prevent variability in the codon AUG recognition.

The choice of using the enzyme beta-glucuronidase (GUS) encoded by the gene uidA as reporter protein was determined by the fact that in tobacco no native GUS-like activity can be observed, and the level of expression of the transgene uidA can be measured by means of a fluorimetric test (Jefferson et al. 1987 EMBO J., 6, 3901-3907) characterized by considerable sensitivity, accuracy, speed and ease of execution.

The fluorimetric readings relating to the enzymatic GUS activity, measured as described by Jefferson (1987) in plants regenerated after transformation (generation T₁) have shown how the presence of the LL-TCK leader causes a considerable increase in the expression level of the uidA gene (up to 15 times) compared with the original construct.

The analysis of variance allowed to establish that the differences found between the two populations of tobacco considered (transformed with pSTART and pBI121) are statistically significant, as are the differences between the best transformants of the two groups.

In order to exclude effects deriving from epigenetic variations, the analysis was repeated on the T₂ progenies obtained from the self-fertilization of the best primary transformants. In this case too, the plants transformed with pSTART showed expression levels for the uidA gene that were much higher than those obtained with pBI121. In particular, an increase equal to 8.6 times was seen in the activity, considering all the plants in their entirety, and equal to 12.5 times considering only the above-average transformants.

In order to determine the effect of LL-TCK on the transcription of the uidA gene, T₂ plants were selected (10 plants for pBI121 and 13 plants for pSTART) characterized by intermediate GUS levels for the analysis of the transcript levels by means of real-time RT-PCR. Starting from the total RNA extracted from each plant, the cDNA used as the template in real-time RT-PCR was synthesized. Two pairs of primers were used (one specific for the uidA gene and one for the endogenous gene of the 18S RNA) and SYBR-Green PCR Master Mix (Applied Biosystems). The correct quantification was made possible by making 2 calibration lines (one for the transgene and one for the endogenous gene) by means of serial dilutions of control plasmids. The transcription level of the uidA gene was then calculated in relative terms for each sample, by means of the percentage ratio between the quantity of mRNA detected for the transgene and the corresponding quantity of ribosomal 18S RNA.

This analysis allowed to verify in the pSTART plants an average transcript level for the uidA gene 1.7 times higher than that found in the pBI121 plants.

For 7 pairs of pSTART and pBIl2l plants characterized by similar transcript values, the TEI (translational efficiency index) was calculated. The TEI is equivalent to the ratio between the GUS protein value measured with the fluorimetric assay and the relative standardized mRNA value determined by the real-time RT-PCR. By comparing the TEI, it is clear that the new LL-TCK leader not only has an effect on the mRNA levels, but also causes an increase in the translation efficiency of the mRNA.

The LL-TCK sequence allows to increase the expression level of a heterologous protein by acting both on the level of the mRNA content relating to the gene concerned, and also on the level of the final quantity of protein present.

The LL-TCK effect was studied in tobacco, using the constitutive CaMV 35S promoter and the uidA gene encoding for the enzyme beta-glucuronidase (GUS), but other uses are possible, in combination with other promoters and other genes.

Although in the examples herein reported the LL-TCK leader is used in combination with the CaMV 35S promoter for enhancement of uidA expression in tobacco plants, said leader was successfully used also in tobacco and potato downstream the light-inducible rbcS1 promoter (GenBank Acc. No. AY163904) and in rice downstream the endosperm-specific, phase-dependent GluB-4 promoter (GenBank Acc. No. AY427571). The genes used in these experiments were those encoding the murine BCL1 antibody, the human acid beta-glucosidase and a synthetic elastin-like polymer. Since no loss of functionality was recorded in experiments carried out with unrelated genes characterized by a different length, base composition and structure put under the control of promoters with a disparate transcriptional activity and expressed in dicot as well as monocot species, it can be stated that the utility of the LL-TCK leader or similarly composed 5′-UTRs is general, that is, not limited to combinations with certain promoters and/or coding sequences and not limited to certain host species. Therefore, the preferred embodiments of the present invention are comprised in a range of biotechnological applications, including the resistance to biotic/abiotic stresses and herbicides, the production of biofuels, bioplastics, synthetic biopolymers and industrial enzymes, the molecular farming of biopharmaceuticals (e.g. antibodies and their fragments, vaccines, human enzymes, cytokines and growth factors), the improvement of food, feed and fiber quality, the development of reporter and marker gene systems.

Furthermore, it comes within the field of the present invention to construct, inside plant expression vectors, 5′-UTRs in which the following elements are simultaneously present: CaMV 35S Inr site, poly(CAA)n, octamer ACAATTAC, poly(CT)n, where n is any number greater than or equal to 2.

All the possible combinations of the elements that constitute the leader sequence 5′-UTR as expressed above, or in the relative variants, irrespective of their relative positioning 5′-3′, come within the field of the present invention.

Furthermore, the present invention also concerns the sequences complementary to those described above, or the relative variants.

According to a variant, the sequence according to the present invention has a length comprised between 20 and 200 nucleotides, preferably between 40 and 150 nucleotides.

According to a variant, the sequence according to the present invention has a G+C content of less than 60%, preferably less than 50%.

One or more amplification primers also come within the field of the present invention, comprising a nucleotide sequence selected from a group comprising the nucleotide sequences shown in SEQ ID NOS: 2-7 or a complement thereof.

According to another form of execution of the present invention, the sequence according to the present invention can be obtained by:
a) artificial synthesis;
b) natural or induced processes of recombination or mutation inside natural or artificial sequences.

One feature of the present invention also concerns a method for the artificial synthesis of a sequence as described above, using one or more of said amplification primers.

The natural 5′-UTR leader sequences that may be discovered and that appear, to a person skilled in the art, to be non-significant variants, provided they are functionally similar, of the sequence according to the present invention, also come within the field of the present invention.

Sequences deriving from mutation processes of the sequence according to the present invention which appear, to a person of skill, to generate non-significant variants, provided they are functionally similar, of the sequence according to the present invention, are also part of the present invention; the mutations concern irrespectively deletions, insertions, transitions, transversions of one or more nucleotides in the sequence according to the present invention or in the sequence complementary thereto.

The present invention also concerns the bacterial strains carrying plasmids containing the sequence according to the present invention, with particular reference to the species Escherichia coli, Agrobacterium tumefaciens and Agrobacterium rhizogenes.

The present invention also concerns engineered bacterial strains containing the sequence according to the present invention, irrespective of the type of host organism.

Furthermore, plant cells transformed with expression vectors containing the sequence according to the present invention under the control of a constitutive promoter also come within the field of the present invention.

The following are also covered by the present invention:
plant cells transformed with expression vectors containing the sequence according to the present invention under the control of a tissue-specific promoter and in particular seed-specific;
plant cells transformed with expression vectors containing the sequence according to the present invention under the control of an inducible promoter;
plant cells transformed with expression vectors containing the sequence according to the present invention under the control of a promoter with phase-dependent transcriptional activity;
plant cells transformed with expression vectors containing the sequence according to the present invention under the control of a promoter active in the chloroplast;
plant cells transformed with expression vectors containing the sequence according to the present invention under the control of a promoter active in the mitochondrion.

The present invention also comprises plants characterized by the transient expression of any protein whose messenger RNA contains the sequence according to the present invention, transient expression being taken to mean the production of said protein by means of viral vectors, agroinfiltration, electroporation, particle delivery.

The present invention also concerns dicotyledonous plants, with particular reference, but not exclusively, to the species belonging to the families of Solanaceae, Papilonaceae and Cruciferae, stably transformed with expression vectors containing the sequence according to the present invention, and also the progenies of said dicotyledonous plants.

The present invention also concerns monocotyledonous plants, with particular reference, but not exclusively, to the species belonging to the family of Graminaceae (Poaceae), transformed with expression vectors containing the sequence according to the present invention, and also the progenies of said monocotyledonous plants.

The present invention has an advantageous industrial application, since it also concerns the use of the sequence according to the present invention for one or another of the following activities:
the biotechnological production of molecules;
the synthesis of recombinant proteins;
the synthesis of recombinant proteins intended to induce resistance to viral, bacterial or fungal pathogens;
the synthesis of recombinant proteins intended to induce resistance to herbicides;
the synthesis of recombinant proteins intended to obtain an altered composition in fatty acids in the raw material and products deriving therefrom;
the synthesis of recombinant proteins intended to obtain an altered nutritional value of the raw material and products deriving therefrom;
the synthesis of recombinant proteins intended for the production of fuels, rubbers and bioplastics;
the synthesis of industrial enzymes and commercial proteins;
the synthesis of pharmaceutical proteins;
the synthesis of orally administered vaccines, intended for men and animals;
the synthesis of injectable vaccines, intended for men or animals;
the synthesis of patient-specific injectable vaccines, preferably idiotype-specific, to be used in treating tumors of the lymphatic system;
the synthesis of proteins involved in the production of secondary metabolites;
the synthesis of proteins used directly or indirectly as factors to identify and/or select transformed cells.

### BRIEF DESCRIPTION OF THE DRAWINGS

These and other characteristics of the present invention will become apparent from the following description of some preferential forms of embodiment, given as a non-restrictive example with reference to the attached drawings wherein:

FIG. 1 is a comparison between the leader sequences in pBI121 and in pSTART, wherein the transcription start site is underlined. Since the sequences between the Eco RV site and the transcription start site and between the Xba I site and the uidA ATG triplet are identical in pSTART and in pBI121, they have been partly omitted (dots);

FIG. 2 shows the expression levels of beta-glucuronidase (GUS) in the transgenic T₁ plants;

FIG. 3 shows the expression levels of beta-glucuronidase (GUS) in the transgenic T₂ plants; the plants are grouped into four groups, each representing sister plants which descend from the best T₁ transformants. Least Significant Difference (P=0.01)=4.7 U/mg of total protein;

FIG. 4a shows the relative transcript levels of uidA (gusA) as determined by real-time RT-PCR in T₂ plants obtained with pSTART and pBI121 characterized by intermediate beta-glucuronidase expression levels. The seven pairs of plants with similar transcript levels are identified;

FIG. 4b shows the values of the translation efficiency index (TEI) for the T₂ plants with similar transcript levels. TEI was calculated as follows: for each transformant, the concentration of beta-glucuronidase (GUS) [U/mg of total protein] was divided by the relative standardized level of mRNA; the highest TEI was considered equal to 1.00 and the values recorded for each transgenic plant were expressed accordingly;

FIG. 5 is a diagram of the overlapping of reverse and forward primers for the synthesis of LL-TCK by means of recursive PCR.

FIG. 6 shows the enzymatic activity of beta-glucuronidase (GUS) in transgenic plants harboring the LL-TCK and the pBI 121 leaders, respectively.

FIG. 7 shows the enzymatic activity of beta-glucuronidase (GUS) in transgenic plants harboring variant ML7.

FIG. 8 shows a comparison between expression levels of the reporter gene (GUS) recorded in primary transformants harboring the ML7 or the LL-TCK leader.

FIG. 9 shows the enzymatic activity of beta-glucuronidase (GUS) in transgenic plants harboring variant ML6.

FIG. 10 shows a comparison between expression levels of the reporter gene (GUS) recorded in primary transformants harboring the ML6 or the LL-TCK leader.

FIG. 11 shows the enzymatic activity of beta-glucuronidase (GUS) in transgenic plants harboring variant ML4.

FIG. 12 shows a comparison between expression levels of the reporter gene (GUS) recorded in primary transformants harboring the ML4 or the LL-TCK leader.

FIG. 13 shows a comparison of expression levels of the reporter gene (GUS) recorded for each of the three tested variant and LL-TCK leader.

### DETAILED DESCRIPTION OF THE PRESENT INVENTION

A) Synthesis of the Artificial Leader Sequence LL-TCK Shown in SEQ ID NO: 1.

A.1) The synthesis of the LL-TCK sequence or similarly composed 5′ -UTRs can most conveniently be achieved by artificial synthesis, making use of specialized services available on the market. Due to the limited length of the sequence, it is especially useful to add at each side of the leader a flanking region ending with a restriction site already present within the promoter sequence (5′ flanking region) and the coding sequence (3′ flanking region). It is obvious to a person skilled in the art that these flanking regions will precisely reproduce the sequence upstream the initiator site (Inr) and the coding sequence, respectively, unless a modification of the promoter and/or the coding sequence is concurrently planned.

A.2) Another procedure to obtain said leader sequence is recursive PCR (Podromou and Pearl 1992 Protein Eng., 5, 827-829, Wheeler et al. 1996 Gene, 169, 251-255, Prytulla et al. 1996 FEBS Letters, 399, 283-289).

Once the LL-TCK sequence or a similarly composed 5′-UTR is obtained by either method, leader variants can easily be produced by PCR or any other procedure developed for random or in situ mutagenesis.

In this example, the description of the LL-TCK synthesis by recursive PCR for its insertion into pBI121 (GenBank accession no. AF485783), in particular between the CaMV 35S promoter and the uidA coding sequence, is reported.

Five synthetic oligonucleotides were used as primers, having a length comprised between 42 and 54 nt and a partial overlapping degree equal to 24 nt, and a terminal reverse primer of 19 nt, shown respectively in the sequences SEQ ID NOS: 2, 3, 4, 5, 6 and 7.

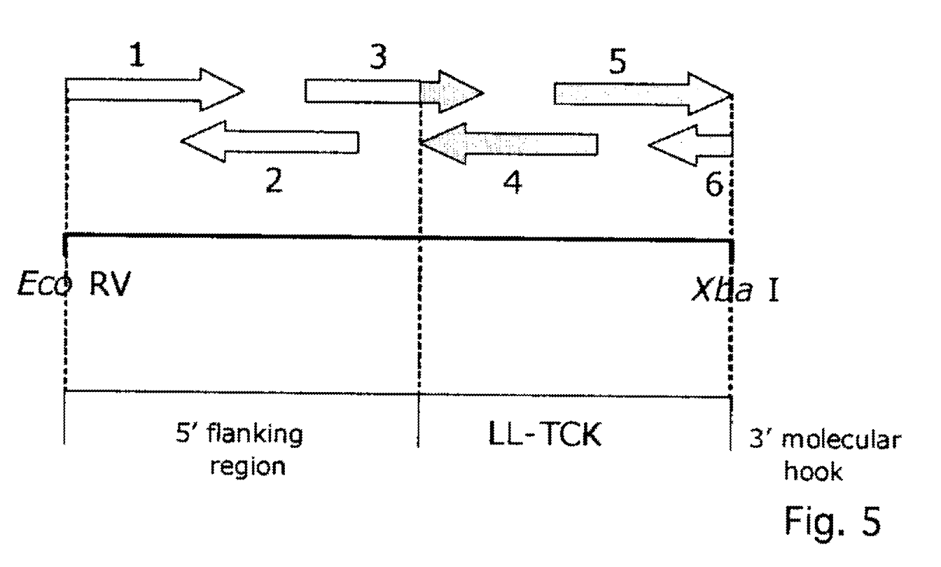

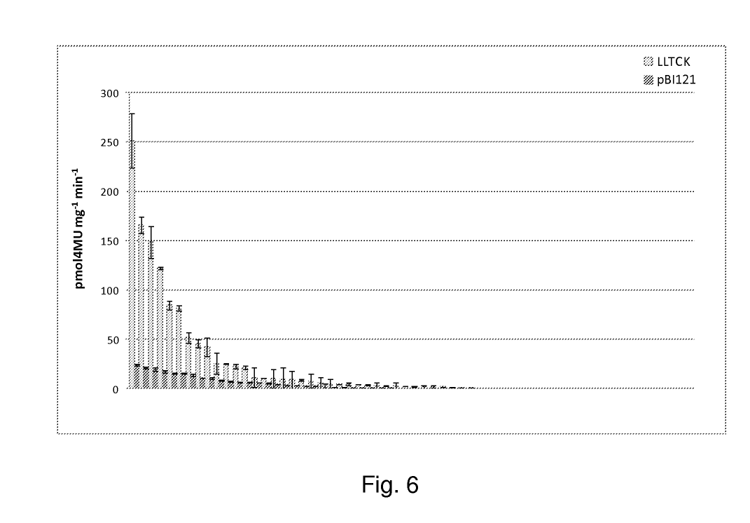

All primers are written in the 5′-3′ direction. The sequences SEQ ID NOS: 2, 4, 6 are forward primers, while the sequences SEQ ID NOS: 3, 5 and 7 are reverse primers. Forward and reverse primers overlap each other according to the diagram in FIG. 5.

To facilitate the handling and the insertion of LL-TCK into the vector sequence concerned, a terminal portion starting with an Eco RV site was added in 5′, while a single Xba I site was added to the 3′ edge.

Therefore, the primers were designed so as to provide the reconstruction of the portion 3′-terminal of the promoter 35S (from the Eco RV site to the Inr region) in order to facilitate the subsequent insertion into the vector pBI121 (Clontech).

The external reverse primer introduces the Xba I site to the terminal 3′, whereas in 5′ the Eco RV site is used.

In pBI121, these sites fall inside the CaMV 35S promoter and in proximity with the translation start signal of uidA, respectively. Therefore, the synthesis was provided of the desired sequence and the cloning to replace the fragment [Eco RV-Xba I].

The primers comprising the nucleotide sequences shown in SEQ ID NOS: 2-7 were designed and created to confirm the promoter sequence in the region between the Eco RV site and the initiator site of CaMV 35S, to synthesize the LL-TCK leader and provide a molecular hook to the terminal 3′ for cloning.

The synthesis of LL-TCK was performed by a single PCR, using a PCR reaction mixture in which the concentration of the external primers, SEQ ID NOS: 2 and 7 (corresponding to the two ends of the synthesized segment) was 100 times greater than that of the internal primers SEQ ID NOS: 3, 4, 5 and 6.

In order to achieve a higher fidelity in DNA synthesis, a proof-reading DNA polymerase was used in combination with a 50% reduction of dNTPs concentration.

The PCR reaction mixture is as follows:
10× Pfu Buffer containing 15 mM Mg^{2+: 10} microL
Primer SEQ ID NO: 2 [10 microM]: 2 microL
Primer SEQ ID NO: 3 [0.1 microM]: 2 microL
Primer SEQ ID NO: 4 [0.1 microM]: 2 microL
Primer SEQ ID NO: 5 [0.1 microM]: 2 microL
Primer SEQ ID NO: 6 [0.1 microM]: 2 microL
Primer SEQ ID NO: 7 [10 microM]: 2 microL
Pfu DNA polymerase [3 U/microL]: 0.8 microL
dNTPs [2.5 milliM each]: 4 microL
Water to a final volume of 100 microL.

In particular, for DNA synthesis and amplification, the DNA polymerase Pfu (Promega) was used, and the following cycle: 1×(95° C. for 5 min); 40×(95° C. for 15 sec; 48° C. for 30 sec; 72° C. for 20 sec); 1×(72° C. for 7 min).

The PCR product was purified by ethanol precipitation, electrophoresed in a 1% agarose gel in TAE buffer, recovered from gel with the aid of a commercial kit, A-tailed with AmpliTaq Gold™, and ligated into pGEM®-T (Promega) for sequencing on both strands.

The ligation mixture was used to transform competent cells of Escherichia coli, strain JM101. The absence of any mismatch between the cloned and the designed sequence was verified by sequencing on double strand.

B) Construction of a Plant Expression Vector Harboring the LL-TCK Sequence.

The possible addition of flanking regions or molecular hooks to the LL-TCK sequence or similarly composed 5′-UTRs offers a broad range of cloning solutions in expression vectors of different kind In this example, the method used to clone the [Eco RV-Xba I] fragment of Example 1 in substitution of the [Eco RV-Xba I] fragment of pBI121 (GenBank accession no. AF485783) is described. Since pBI121 has multiple Eco RV sites beyond that inside the CaMV 35S promoter, the latter promoter was excised from pBI121 (Clontech) (Jefferson et al. 1987) making use of the Hind III and Xba I restriction enzymes.

The fragment was recovered from 1% agarose gel in TAE buffer and subcloned in pUC18 (Pharmacia, GenBank accession no. L08752), previously digested with the same enzymes.

As we said, this passage was necessary because pBI121 has multiple Eco RV sites. The pUC18/35S vector obtained was used to make the new combination of 35S promoter—LL-TCK leader.

The LL-TCK sequence was excised from the pGEM-T vector by means of digestion with Eco RV and Xba I (NEB), separated from the vector sequence by agarose gel electrophoresis and subsequently recovered from gel with the aid of a commercial kit. The pUC18/35S vector was in turn digested with the same enzymes, treated with alcaline phosphatase (Pharmacia), electrophoresed and recovered from gel as above. Then a ligation reaction was carried out at 4° C. for 16 hours in the presence of T4 DNA ligase (Promega). In particular, 3.5 ng of the [Eco RV-Xba I] fragment were combined with 25 ng of the vector in the presence of 1 U T4 DNA ligase in a volume of 10 microL containing a suitable reaction buffer.

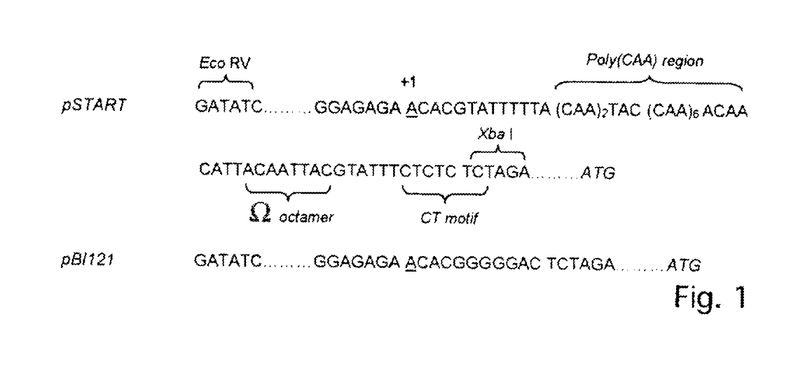

The pBI121 vector (Clontech) was subjected to digestion with Xba I and Hind III (NEB) to remove the CaMV 35S promoter. The complex 35S-LL-TCK was in turn excised from the cloning vector pUC18 by means of digestion with the same enzymes; the pBI121 vector frame and the 35S-LL-TCK insert were electrophoresed and recovered from gel as above. Finally, a ligation of 35S-LL-TCK in the pBI121 framework was performed, obtaining the vector pBI121/35S-LL-TCK:: uidA::NOS to which the name pSTART (FIG. 1) was assigned.

C) Transformation of Plants with an Expression Vector Containing the LL-TCK Sequence.

Transgenic plants harboring the LL-TCK sequence or similarly composed 5′-UTRs can be produced through a range of methods, including coinfection with engineered strains of Agrobacterium spp., infection or trasfection with engineered strains of phytoviruses, electroporation, particle delivery, DNA microinjection.

The pSTART expression vector was electroporated into Agrobacterium tumefaciens strain EHA 105 and the transformed Agrobacterium cells used for tobacco (Nicotiana tabacum L., cv. Xanthi) transformation. Briefly, 2 mL LB medium supplemented with kanamycin (50 mg/L) were inoculated with transformed Agrobacterium cells. Bacterial cultures were incubated at 29° C. for 16 hours. Leaf discs (7 mm in diameter) were obtained with a cork borer from axenically grown, 30-d old seedlings or from mature leaves collected from plants at the late-rosette stage. In the latter case, tobacco leaves were rinsed with distilled water, surface-sterilized in 1% sodium hypochlorite for 5 min and in 95% ethanol for 30 sec and blotted to sterile filter paper under a laminar flow hood.

Leaf discs were placed in a Petri dish containing 15 mL of Murashige and Skoog medium supplemented with 0.1 mg/L naphthalene acetic acid (NAA), 1 mg/L 6-benzyladenine (BA), 30 g/L sucrose and solidified with 8 g/L agar. Immediately after this transfer, 2 mL of the above-mentioned Agrobacterium culture were poured in the Petri dish and the leaf discs were uniformly wetted. After removal of the LB medium in excess, the dish was sealed and incubated for 24 hours at 25° C. in the light (30.5 microE/square meter/sec).

Leaf discs were then transferred to a new Petri dish containing 15 mL of Murashige and Skoog medium supplemented with 0.1 mg/L naphthalene acetic acid (NAA), 1 mg/L 6-benzyladenine (BA), 500 mg/L cefotaxime, 30 g/L sucrose and solidified with 8 g/L agar.

They were incubated for a week at 28° C. and illuminated for 16 hours/day; they were eventually transferred to a substrate identical to the former except for the presence of 200 mg/L kanamycin. Explants were subcultured every 3 weeks; regenerated shoots were isolated from callus tissue and rooted on semisolid Murashige and Skoog medium supplemented with 2 mg/L indole-3-butyric acid, 500 mg/L cefotaxime, 200 mg/L kanamycin, 30 g/L sucrose.

Putative transgenic plants were potted in peat and grown in a greenhouse under Powerstar HQI-T lamps (Osram) (200 mM photons/square meter/sec at canopy level) for 16 hours/day at 25° C./19° C. light/dark.

In this example, transformation was confirmed by PCR and beta-glucuronidase assays. For PCR assay, total DNA was extracted according to Doyle and Doyle (1990) and the following primers were used:

(SEQ ID NO: 8)
Forward 5'-ACAATTACGTATTTCTCTCTCTAGA-3'
(SEQ ID NO: 9)
Reverse 5'-CGATCGGGGAAATTCGAGCTC-3'

The forward and reverse primers anneal to the end of the LL-TCK sequence and to part of the NOS terminator, respectively and do not give rise to any amplification product in untransformed Xanthi plants (in transgenic plants the amplicon length is 1936 bp, as expected).

About 93% of the regenerated plants were found transgenic when a standard reaction mix was formed and the following temperature cycling was used: 1×(94° C. for 5 min); 40×(94° C. for 1 min 15 sec; 60° C. for 45 sec; 72° C. for 2 min); 1×(72° C. for 5 min).

Plant transformation was further demonstrated by GUS histochemical assays (Jefferson et al. 1987) and fluorometric determination of GUS activity. Controls consisted of Xanthi plants raised in vitro from uninfected discs. The methods used for the fluorimetric assay and the results obtained are reported in detail in point D.

The same procedure was followed to produce and characterize transgenic plants harboring the original leader sequence. Ceteris paribus, no effect of the LL-TCK sequence was noted upon regeneration and transformation rates.

D) Effect of the LL-TCK Sequence on Transgene Expression Levels.

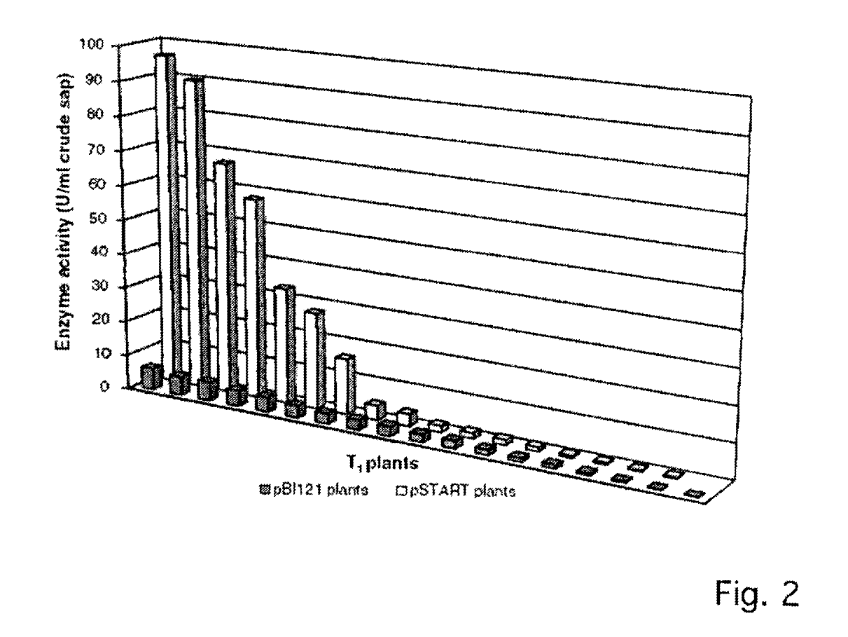

As previously indicated, the plasmids pBI121 and pSTART were individually used for Agrobacterium-mediated transformation of tobacco leaf discs. Since in both cases the gene under control is uidA (also known as gusA), transgene expression levels achievable with LL-TCK and the widely-distributed pBI121 leader can be compared directly by determining the activity of the uidA encoded enzyme, beta-glucuronidase-(EC 3.2.1.31). About twenty primary transformants (that is transgenic plants belonging to the first generation, T₁) of each population were assayed for transgene presence by PCR and subsequently analyzed for beta-glucuronidase activity (FIG. 2). When the late rosette stage was reached (30 days upon hardening), the 3 youngest leaves were collected from each plant to obtain crude sap by pressing (Erich Pollähne); 100 microL of crude sap were mixed with 2 volumes of extraction buffer (Jefferson 1989) containing 12 mg of high molecular weight polyvinyl pyrrolidone (PVP). After centrifugation for 15 min at 11,500×g, the supernatant was collected and analyzed fluorometrically (Dyna Quant 200 fluorometer; GE Healthcare) in duplicate using 4-methylumbelliferyl-beta-D-glucuronide (MUG; Sigma-Aldrich) as substrate. Trials were made to determine the background noise due to intrinsic fluorescence of the samples, quenching, as well as substrate degradation by factors other than the recombinant enzyme. The level of transgene expression was measured in terms of beta-glucuronidase units per mL of crude sap, one unit being defined as the amount of enzyme releasing 1 nM 4-methylumbelliferone (4-MU) min⁻¹ using the same assay conditions as previously described (Jefferson 1989). Data were submitted to a log transformation to avoid any correlation between variance and mean; the analysis of variance was carried out after checking the normality of the distribution of log data by means of the Kolmogorov-Smirnov test and the homogeneity of variances with Bartlett's formula. Averages were compared with Duncan's multiple range test at the probability level, P=0.05.

The analysis of variance carried out on fluorescence data showed the absence of any significant variation among young leaves of the same plant, whereas remarkable differences existed among plants. Specifically, the synthetic leader determined a highly significant increase (up to 15-fold) in uidA expression (Table 1).

TABLE 1
Beta-glucuronidase activity (U/mL of crude sap) in randomly-
chosen T₁ plants

| pSTART | pBI121 |
|---|---|
| 96.10 | 6.24 |
| 89.81 | 5.37 |
| 67.06 | 5.34 |
| 57.89 | 4.52 |
| 33.16 | 4.20 |
| 27.62 | 3.55 |
| 15.97 | 2.95 |
| 4.24 | 2.89 |
| 3.51 | 2.79 |
| 1.85 | 2.34 |
| 1.64 | 2.30 |
| 1.46 | 1.62 |
| 1.21 | 1.17 |
| 0.77 | 1.12 |
| 0.64 | 0.75 |
| 0.46 | 0.47 |
| 0.38 | 0.23 |
| | 0.15 |

To demonstrate that these outcomes were not biased by epigenetic variation, analyses were repeated on T₂ progenies. In particular, the best 4 T₁s in each population were selfed and the resulting seed plated on a kanamycin-enriched medium for selection; 5-7 resistant T₂ plants were randomly chosen within each progeny and raised to the late rosette stage for verification of the transgene presence by PCR and measurement of beta-glucuronidase activity by fluorometric assays; enzyme levels were again expressed as units per mL of crude sap but also as units per mg total protein (as determined by Bradford assay) to account for plant-to-plant variation in metabolism.

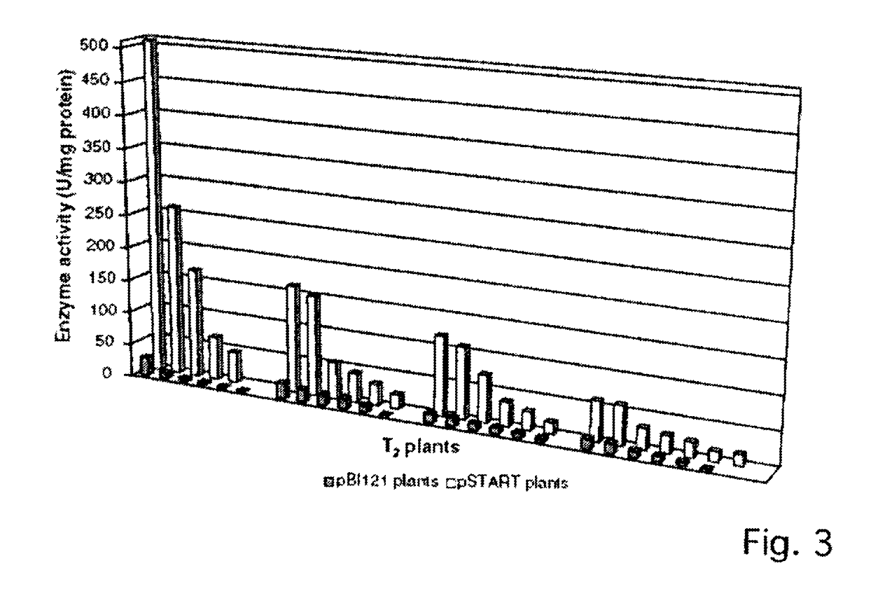

Similarly to what observed in the T₁ generation, transgenic T₂ plants harboring the new leader showed significantly higher levels of uidA expression (FIG. 3); as compared to the pBI121 leader, a 8.6-fold and a 12.5-fold increase of activity were estimated taking into account the whole plant population or the above-average transformants, respectively (Table 2).

TABLE 2
Beta-glucuronidase activity (U/mg protein) in T₂ plants obtained from the
best 4 T₁s.

| pSTART | pBI121 |
|---|---|
| 139.07 | 789 |
| 71.27 | 5.17 |
| 45.43 | 4.44 |
| 44.81 | 3.96 |
| 41.67 | 3.94 |
| 32.92 | 3.78 |
| 29.81 | 3.77 |
| 19.79 | 3.58 |
| 18.04 | 3.55 |
| 16.40 | 3.23 |
| 15.10 | 3.15 |
| 13.55 | 3.09 |
| 12.84 | 2.94 |
| 12.32 | 2.62 |
| 10.78 | 2.45 |
| 10.02 | 1.71 |
| 9.00 | 1.28 |
| 8.50 | 1.22 |
| 6.98 | 1.14 |
| 6.57 | 0.64 |
| 5.39 | 0.52 |
| 5.16 | 0.46 |
| 5.01 | 0.44 |
| 3.17 | 0.31 |

E) Effect of the LL-TCK Sequence on Gene Transcription and Translation.

The combination of different elements in LL-TCK or similarly composed 5′-UTRs reflects in measurable improvements of transcriptional as well as translational efficiency of a given transgene. In this example, such improvements are shown in transgenic T₂ tobacco plants obtained with pBI121 and pSTART, as described before. In order to collect such evidence, plants belonging to the pBI121 or the pSTART group (10 and 13 T₂s, respectively) and characterized by intermediate uidA expression levels were analyzed to determine:

i. the mean transcript levels of uidA;

ii. the mean transcript levels of 18S RNA;

iii. the amount of beta-glucuronidase actually produced.

To minimize experimental error, one young leaf was collected from each plant and cut in two, one half being used for RNA isolation, the other for beta-glucuronidase assay.

Total RNA was extracted with RNAgents Total RNA Isolation System (Promega). First-strand cDNA was synthesized from 1 microg RNA by AMV Reverse Transcriptase (Promega) in the presence of random primers. The cDNA synthesis reaction was diluted 1:5 and 1 microL was used for real-time PCR (qRT-PCR).

qRT-PCRs were performed making use of SYBR-Green PCR Master Mix (Applied Biosystems) and specific primers each at a 0.3 microM final concentration. All reactions were carried out with the iCycler iQ multicolor real-time PCR detection system (Bio-Rad) and run with the following program: 1×(95° C. for 10 min); 50×(95° C. for 15 sec; 62° C. for 30 sec; 72° C. for 30 sec). To amplify the uidA transcript, the following primers were used:

(SEQ ID NO: 10)
Forward 5'-TTACGCTGAAGAGATGCTCGAC-3'
(SEQ ID NO: 11)
Reverse 5'-CCTAAAGAGAGGTTAAAGCCGACAG-3'

For the 18S RNA target sequence, the primers were designed on the basis of GenBank accession n. AJ236016:

(SEQ ID NO: 12)
Forward 5'-ACATCCAAGGAAGGCAGCAG-3'
(SEQ ID NO: 13)
Reverse 5'-GACTCATAGAGCCCGGTATTGTTATT-3'

In both cases, the amplicon length was 90 bp. In each PCR run, serial dilutions of control plasmids were included in parallel with known amounts of input copy number in order to draw standard calibration curves. Specifically, 10-fold serial dilutions (from 10⁵ to 10² copies) of the uidA-harboring plasmid pBI221, were used as templates. For the same purpose, a 550 bp fragment of the 18S RNA gene (AJ236016) was cloned in pGEM-T Easy (Promega) and used in the range of 10⁸ -10⁵ copies.

The starting quantities of uidA transcript and control RNA were determined with ICycler IQ real-time detection system software ver. 3.0. For each sample, at least 3 independent estimates were performed; in all cases, the maximum value for the variation coefficient (VC=SD/mean) was fixed at 20%. The % ratio between the mean transcript levels of uidA and 18S RNA was calculated for each sample. To standardize data, the highest uidA transcript level was considered equal to 1.00 and values recorded for each transgenic plant were expressed accordingly.

Results obtained in qRT-PCR indicated that the substitution of the pBI121 leader with LL-TCK determines a clear increase of average uidA transcript levels. Specifically, the transcriptional efficiency was found 1.7-fold higher in plants harboring the LL-TCK leader (Table 3).

TABLE 3
% ratio between uidA and 18S transcripts in several T₂ plants

| pSTART | pBI121 |
|---|---|
| 0.01371% | 0.00881% |
| 0.01189% | 0.00832% |
| 0.01132% | 0.00761% |
| 0.00968% | 0.00437% |
| 0.00960% | 0.00315% |
| 0.00896% | 0.00308% |
| 0.00578% | 0.00282% |
| 0.00556% | 0.00234% |
| 0.00425% | 0.00225% |
| 0.00315% | 0.00158% |
| 0.00272% | 0.00072% |
| 0.00201% | |
| 0.00189% | |

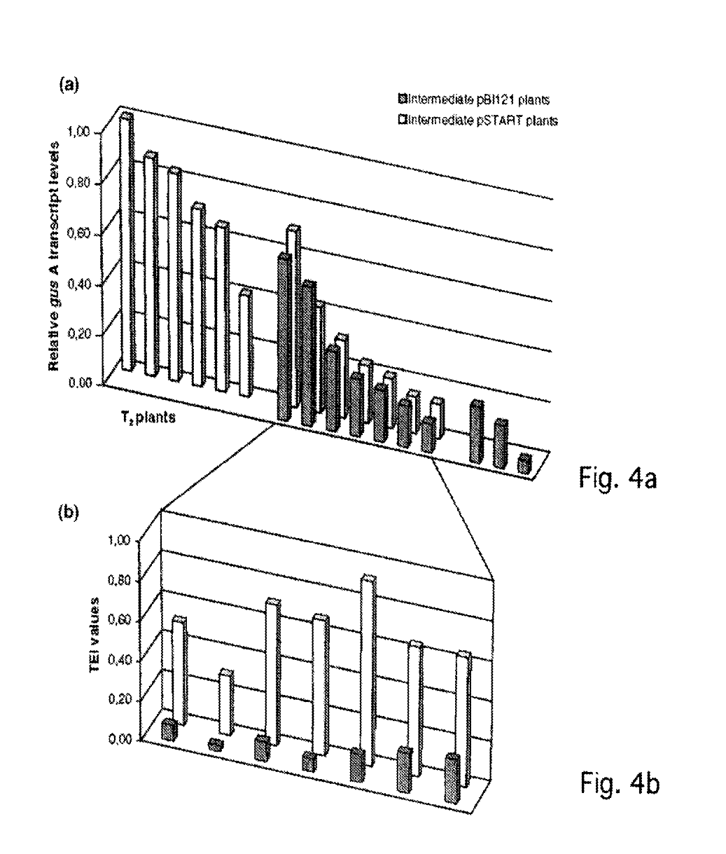

In addition, after having identified 7 plant couples with nearly overlapping uidA transcript levels (FIG. 4a), a translational efficiency index (TEI) was attributed to each plant (FIG. 4b), making the ratio between beta-glucuronidase concentration and the relative standardized level of uidA transcript. By comparison of TEI values, the two leaders were found to determine a clearly different translational efficiency of uidA transcripts, which was greater using the leader according to the present invention (Table 4).

TABLE 4
ratio between GUS enzymatic activity and relative transcription level of
uidA gene measured in some T₂ plants

| TEI | pSTART | pBI121 | TEI |
|---|---|---|---|
| 1.00 | 299.07 | 103.06 | 0.34 |
| 0.93 | 277.07 | 66.58 | 0.22 |
| 0.71 | 211.87 | 58.52 | 0.20 |
| 0.68 | 203.76 | 19.82 | 0.07 |
| 0.65 | 195.24 | 42.97 | 0.14 |
| 0.65 | 194.12 | 47.18 | 0.16 |
| 0.52 | 154.91 | 20.86 | 0.07 |
| 0.34 | 102.01 | 29.52 | 0.10 |
| 0.30 | 89.48 | 8.94 | 0.03 |
| 0.27 | 80.26 | 12.34 | 0.04 |
| 0.25 | 73.69 | 22.81 | 0.08 |
| 0.20 | 59.70 | | |
| 0.17 | 49.40 | | |

### Additional Leader Sequences

Additional leader sequences were produced and tested. The following Table 5 shows such variants and their main differences with respect to the LL-TCK sequence.

TABLE 5
Sequence of LL-TCK and of its variants

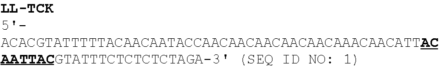

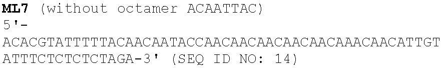

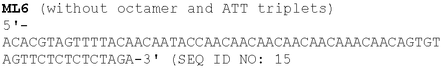

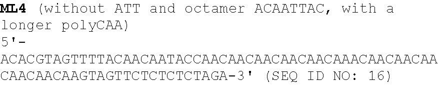

Materials and methods were essentially those illustrated in the examples relating to LL-TCK above, including the reporter gene (uidA). Each variant was synthesized artificially and substituted to LL-TCK in pSTART. For each of them, a population of about 45 transgenic plants was obtained.

To evaluate the new variants of the LL-TCK sequence, a comparison between them and the LL-TCK sequence was made, always analyzing the expression levels of the reporter protein GUS in transformed tobacco plants (Nicotiana tabacum L.).

The expression levels of the reporter gene were assessed using the fluorometric 4-MUG assay (Jefferson et al., 1987), characterized by considerable sensitivity, precision, speed and ease of execution. In particular, to quantitatively evaluate the expression of the uidA gene in transformed plants, fluorometric assays were carried out on crude protein extracts derived from pressing three, completely distended, young leaves. The values of specific activity for beta-glucuronidase (GUS), expressed in millimoles of 4-MU produced per mg of protein, were normalized in relation to the total protein concentration calculated using a Bradford assay. This method was applied to all variants.

Results obtained for each leader sequence are shown below. Moving from variant ML7 to variant ML6 and then to variant ML4, the sequence differences become more pronounced and evident compared to LL-TCK.

Variant ML7 and its Comparison with LL-TCK

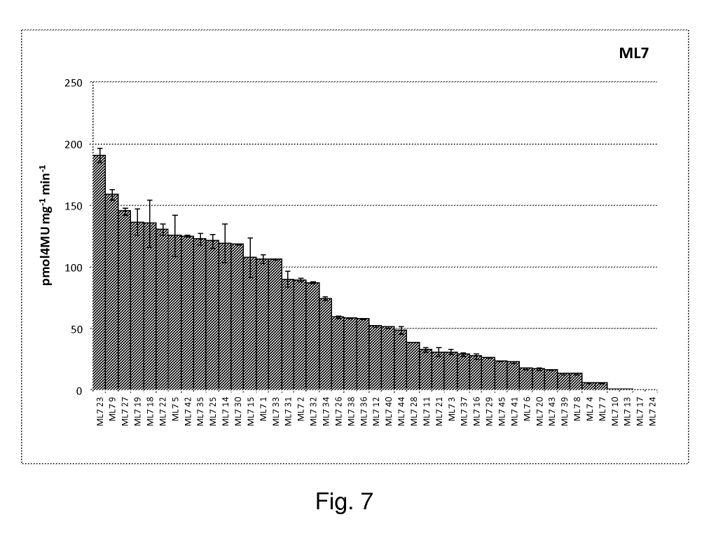

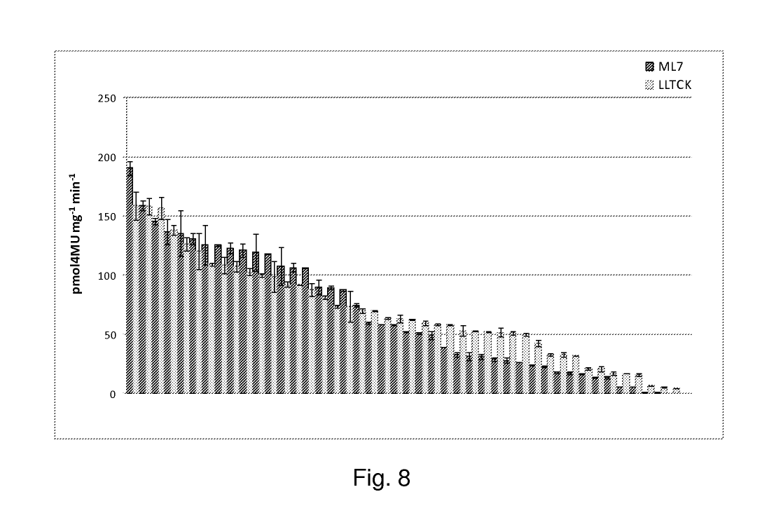

ML7 variant harbors the same repeated CAA trinucleotide elements and repeated CT dinucleotide elements, as LL-TCK sequence, without the octamer ACAATTAC. This example clearly demonstrates that even in absence of this element (octamer ACAATTAC), high levels of protein expression can be reached, as shown in FIG. 7. In fact, the comparison between variant ML7 and LL-TCK demonstrated that protein expression levels are unaffected by the deletion of this element, as shown in FIG. 8.

In particular, two populations, each harboring a different leader and consisting of 45 primary transformants, were produced and analyzed for GUS activity. The Mann-Whitney U test, used for statistical analysis of data, revealed no significant difference between the two populations (p-value=0.603). FIG. 8 shows the data obtained on the transformed plants with the two constructs (average of the values recorded for the three leaf extracts) in decreasing order.

Variant ML6 and its Comparison with LL-TCK

ML6 variant harbors the same repeated CAA trinucleotide elements and repeated CT dinucleotide elements, as LL-TCK sequence, without the octamer ACAATTAC and ATT triplets, replaced by AGT ones.

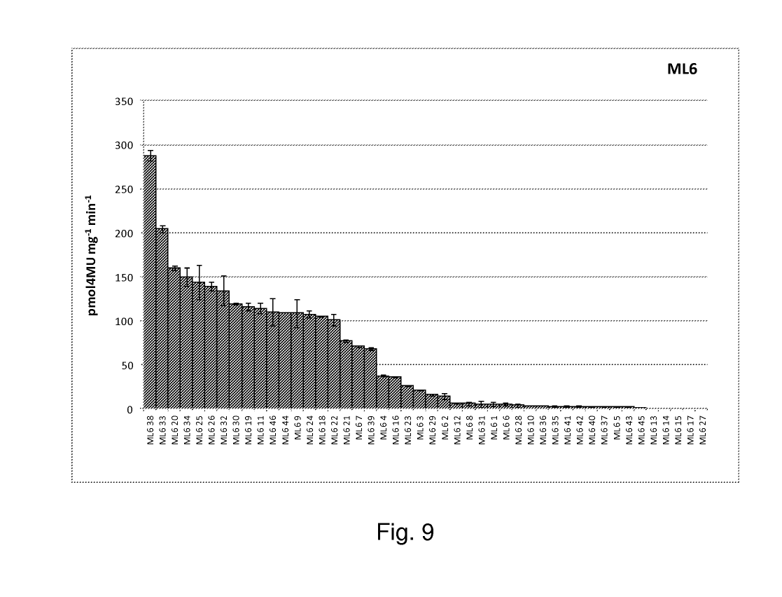

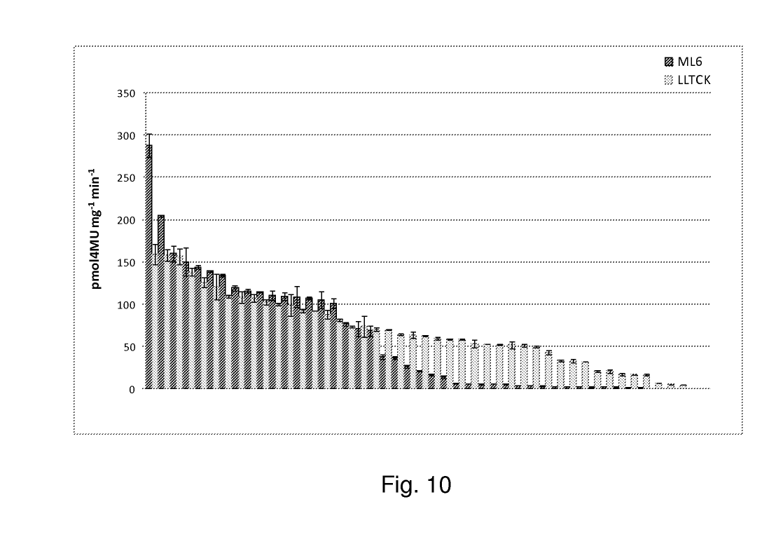

The substitution of ATT triplets was performed to test the possible effect of removal of non-canonical translation start codons (i.e. ATT triplets) in a LL-TCK-derived leader sequence devoid also of the Ω octamer. Since the removal of all ATT triplets would have determined a significant shortening of the leader sequence, a leader was mutated by replacing the “T” nucleotide located in the central position of each ATT triplet with a “G” nucleotide. Experiments were then designed to challenge the mutated leader (variant ML6) alone and versus the LL-TCK leader in terms of induced GUS expression levels. FIG. 9 shows the GUS expression levels obtained with variant ML6, while FIG. 10 shows the comparison between it and LL-TCK. In particular, two populations each consisting of 45 primary transformants harboring the two leaders were produced and analyzed; the Mann-Whitney U test, used for statistical analysis of data, revealed no significant difference between the two populations (p-value=0.063).

Variant ML4 and its Comparison with LL-TCK

ML4 variant harbors repeated CAA trinucleotide elements and repeated CT dinucleotide elements, without the octamer ACAATTAC and ATT triplets, as variant ML6, and with a poly(CAA) longer than LL-TCK.

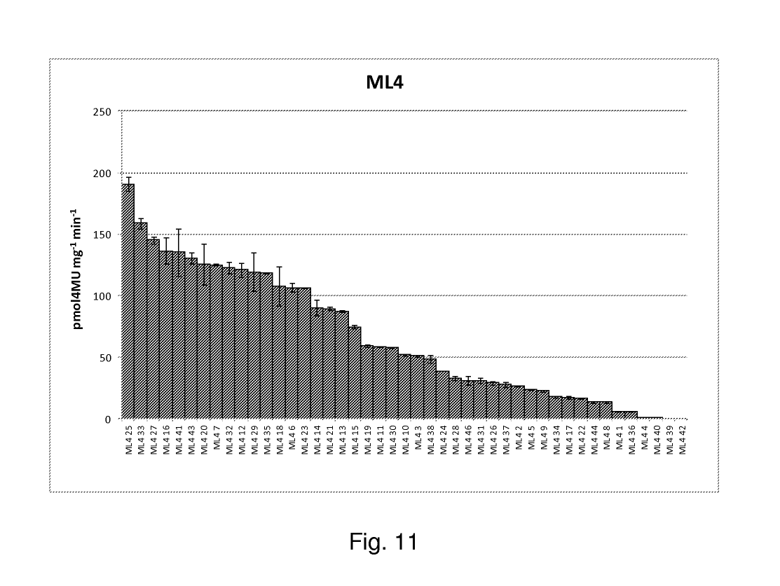

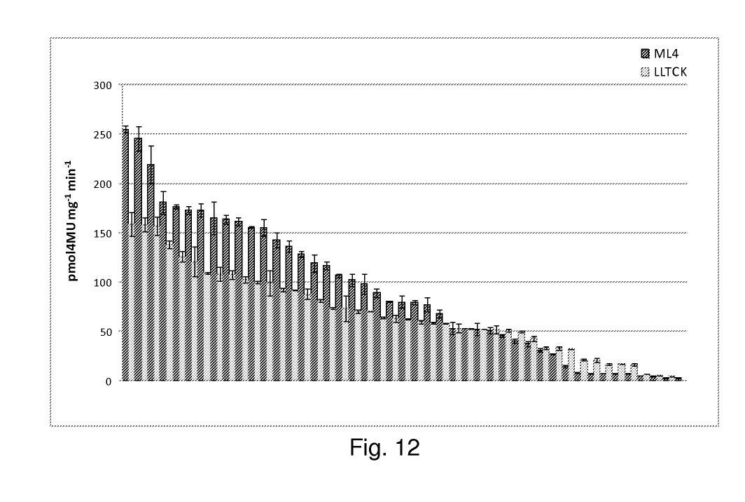

Also in this third case, the variant considered allows to gain interesting level of protein expression (FIG. 11), quite similar to those recorded for LL-TCK. In fact, in the comparison of the two population for GUS expression (FIG. 12), the Mann-Whitney U test, revealed no significant difference between the two populations (p-value=0.224).

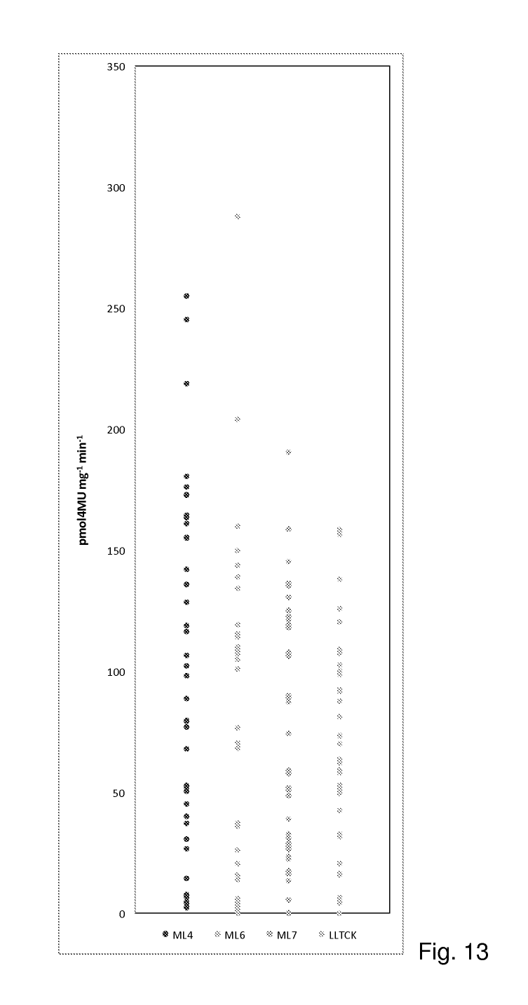

Data collected for each example are summarized in FIG. 13, which gives an overview of the quite similar levels of protein expression achievable with LL-TCK leader and its variants.

1. A nucleotide leader sequence 5′-UTR, comprising a poly(CAA) region of two or more contiguous or non-contiguous CAA trinucleotide elements and a poly(CT) region of two or more contiguous or non-contiguous CT dinucleotide elements.

2. The sequence of claim 1, wherein said poly(CAA) region is located in a position 5′ with respect to said poly(CT) region.

3. The sequence of claim 1 wherein said poly(CAA) region comprises nine CAA trinucleotide elements.

4. The sequence of claim 1 wherein said poly(CT) region comprises four CT dinucleotide elements.

5. The sequence of claim 1, further comprising at least one element selected from the group consisting of the octamer ACAATTAC, and the transcription initiation site of the cauliflower mosaic virus 35S promoter.

6. The sequence of claim 1, wherein the length of said sequence is between 20 and 200 nucleotides.

7. The sequence of claim 1, wherein the length of said sequence is between 40 and 150 nucleotides.

8. The sequence of claim 1, wherein the G+C content of said sequence is less than 60%.

9. The sequence of claim 1, wherein the G+C content of said sequence is less than 50%.

10. The sequence of claim 5, wherein said sequence comprises said poly(CAA) region, said poly(CT) region, the octamer ACAATTAC, and the transcription initiation site of the cauliflower mosaic virus 35S promoter.

11. The sequence of claim 5, wherein said sequence comprises said poly(CAA) region, said poly(CT) region, and the transcription initiation site of the cauliflower mosaic virus 35 S promoter.

12. A plant expression vector comprising a sequence of claim 1 under the control of a promoter.

13. A plant cell transformed with an expression vector of claim 12.

14. A bacterial strain carrying plasmids containing the sequence of claim 1.

15. An engineered virus strain containing the sequence of claim 1.

16. A plant characterized by the transient expression of any protein in which the messenger RNA contains the sequence of claim 1.

17. A dicotyledonous plant or progeny thereof stably transformed with an expression vector of claim 12.

18. A monocotyledonous plant or progeny thereof stably transformed with an expression vector of claim 12.

19. A method for the synthesis of a recombinant protein comprising expressing a nucleotide sequence encoding said protein and located downstream to the sequence of claim 1.

20. A method for the synthesis of a recombinant protein comprising the step of expressing a nucleotide sequence encoding said protein and located downstream to the sequence of claim 1 in a transformed plant cell.
